(19) United States
(12) Patent Application Publication
(10) Pub. No.: US 2009/0061228 A1
(43) Pub. Date: Mar. 5, 2009

(54) METHOD OF PREPARING SOLID PARTICULATES AND SOLID PARTICULATES PREPARED USING SAME

(75) Inventors: Hae-Ryong CHUNG, Suwon-si (KR); Eun-Sang KWON, Sendai-si (JP); Hachiro NAKANISHI, Sendai-si (JP); Hidetoshi OIKAWA, Sendai-si (JP); Hitoshi KASAI, Sendai-si (JP)

Correspondence Address: SUMMA, ADDITON & ASHE, P.A., 11610 NORTH COMMUNITY HOUSE ROAD, SUITE 200, CHARLOTTE, NC, 28277, US

(73) Assignees: CHEIL INDUSTRIES INC., Gumi-si (KR); TOHOKU UNIVERSITY, Sendai-si (JP)

(21) Appl. No.: 12/204,144

(22) Filed: Sep. 4, 2008

(30) Foreign Application Priority Data
Sep. 5, 2007 (KR) 10-2007-0090174

(51) Int. Cl. B32B 5/16; C07C 15/02

(52) U.S. Cl. 428/402; 585/400

(57) ABSTRACT

The present invention relates to a method of preparing solid particulates and solid particulates prepared by using the method. The method of preparing solid particulates includes dissolving an organic or inorganic compound in a first solvent to provide an organic or inorganic compound-included solution, dispersing the organic or inorganic compound-included solution in a second solvent to provide an emulsion, and concentrating the emulsion in a dispersing medium to precipitate the organic or inorganic compound as solid particulates to provide a dispersion including the solid particulates. The first solvent is an organic solvent or an aqueous solvent, and the second solvent is an organic solvent or an aqueous solvent that is not compatible with the first solvent. It is possible to prepare solid particulates from a wide range of organic or inorganic compounds in accordance with the present invention, and in addition, it is possible to prepare a dispersion including organic or inorganic compound particulates in a high concentration. Therefore, according to the method of the present invention, the solid particulates can be mass-produced.

### CROSS-REFERENCE TO RELATED APPLICATION

This application claims priority to and the benefit of Korean Patent Application No. 10-2007-0090174 filed in the Korean Intellectual Property Office on Sep. 5, 2007, the entire disclosure of which is incorporated herein by reference.

### FIELD OF THE INVENTION

The present invention relates to a method of preparing solid particulates and solid particulates prepared using the same.

### BACKGROUND OF THE INVENTION

Nano-sized particulates or micro-grain particles express particular physical properties due to the quantum size effects. Accordingly, much research on methods of preparing nano-sized particulates or micro-grain particles has been undertaken.

For example, Japanese Patent Publication Nos. 2723200 and 3423922 disclose a re-precipitation as a method of preparing organic particulates. The re-precipitation is a method of generating organic compound particulates by injecting a solution in which an organic compound is dissolved in a good solvent into a poor solvent (commonly, distilled water) by using a syringe. However, in this method, the good solvent includes only a polar solvent that is capable of being unlimitedly diluted in a poor solvent. Thereby, the organic compound is limited to one that can be dissolved in some kind of polar solvent.

Japanese Patent Laid-open Sho 62-106833 discloses a method of condensing organic compound particulates by heating and evaporating the organic compound contained in a heating container in a vacuum container, and controlling an inert gas pressure in the vacuum container. However, it is not suitable for mass-production because it is difficult to recover the particulates prepared by the method and the vacuum container is required to produce the solid particulates. The organic compound is also limited because it must have a certain vapor pressure.

A practicable method that is capable of application to a wide range of compounds such as organic compounds or inorganic compounds and mass-producing highly concentrated particulates has never been suggested.

### SUMMARY OF THE INVENTION

An exemplary embodiment of the present invention can provide a method of preparing solid particulates that can be used with various organic or inorganic compounds.

The present invention can further provide a method for the mass-production of solid particulates from a variety of compounds including organic and inorganic compounds.

Another embodiment of the present invention provides a method of preparing solid particulates that can be applicable to mass production by preparing organic or inorganic compound particulates in dispersion in a high concentration.

The present invention also provides solid particulates prepared by the method.

The embodiments of the present invention are not limited to the above technical purposes, and a person of ordinary skill in the art can understand other technical purposes.

According to one embodiment of the present invention, provided is a method of preparing solid particulates, including: dissolving an organic or inorganic compound in a first solvent to provide an organic or inorganic compound-included solution; dispersing the organic or inorganic compound-included solution in a second solvent to provide an emulsion; and concentrating the emulsion in a dispersing medium to precipitate the organic or inorganic compound as solid particulates so as to provide a dispersion including solid particulates.

The first solvent is an organic solvent or an aqueous solvent, and the second solvent is either an organic solvent or an aqueous solvent that is not compatible with the first solvent.

According to another embodiment, the organic compound is selected from the group consisting of aromatic cyclic compounds, heterocyclic compounds, fullerenes, and mixtures thereof.

In another embodiment, the inorganic compound is an ionic crystalline compound.

In a further embodiment, the organic solvent has solubility relative to the organic compound ranging from about 1 mass % to a saturated concentration.

In further another embodiment, the aqueous solvent has solubility relative to the inorganic compound ranging from about 1 mass % to a saturated concentration.

In further another embodiment, the second solvent has solubility relative to the solid particulates of about 1.0×10⁻¹ g/L or less at 20° C.

According to another embodiment, the organic or inorganic compound-included solution is mixed with the second solvent at a volume ratio ranging from about 0.01:1 to about 0.7.1.

The method of preparing an emulsion can be performed by a method selected from the group consisting of high-speed agitation of the organic or inorganic compound-included solution, ultrasonic wave radiation, and combinations thereof.

The concentration step can be performed by reducing the pressure of the emulsion to a range of about 0.01 to about 10.0 hPa/min, for example, by reducing the pressure to a range of about 0.1 to about 5.0 hPa/min.

According to another embodiment, the dispersion can include solid particulates in a concentration ranging from about 1.0 to about 1000 μg/ml.

Another embodiment of the present invention provides solid particulates prepared by the method of the invention.

In another embodiment, the solid particulates can have an average diameter ranging from about 10 to about 1000 nm.

According to the method of the present invention, the solvent may include any solvent as long as the solvent can form an emulsion because the required crystals grow in an emulsion. Therefore, it is possible to prepare solid particulates for a wider range of organic or inorganic compounds than with the conventional re-precipitation method of preparing solid particulates. It is possible to use a water-in-oil emulsion or an oil-in-water emulsion, so that it is possible to provide solid particulates of inorganic compounds as required. According to the method of the present invention, it is possible to obtain size-controlled solid particulates in a high concentration from the desirable dispersing medium.

### BRIEF DESCRIPTION OF THE DRAWINGS

FIG. 1 is a scanning electron microscope (SEM) photograph of perylene particulates according to Example 1.

FIG. 2 is a scanning electron microscope photograph of perylene particulates according to Example 2.

FIG. 3 is a scanning electron microscope photograph of perylene particulates according to Example 3.

FIG. 4 is a scanning electron microscope photograph of perylene particulates according to Example 4.

### DETAILED DESCRIPTION OF THE INVENTION

The present invention now will be described more fully hereinafter in the following detailed description of the invention, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

In the present specification, when specific definition is not provided, “alkyl” refers to a C₁-C₂₀ alkyl, “alkoxy” refers to a C₁-C₂₀ alkoxy, and “aryl” refers to a C₆-C₁₈ aryl.

The method of preparing solid particulates according to one embodiment of the present invention includes: dissolving an organic or inorganic compound in a first solvent to provide an organic or inorganic compound-included solution (S1); dispersing the organic or inorganic compound-included solution in a second solvent to provide an emulsion (S2); and concentrating the emulsion in a dispersing medium to precipitate the organic or inorganic compound as solid particulates, so as to provide a dispersion including solid particulates (S3).

Hereinafter, each step of the present invention is described in detail. Firstly, the organic or inorganic compound which is a compound for solid particulates is dissolved in a first solvent to provide an organic or inorganic compound-included solution (S1).

The organic or inorganic compound is not limited as long as it can be dissolved in the first solvent and precipitated to a solid depending upon a solubility decrease thereof.

The organic compound may include aromatic cyclic compounds, heterocyclic compounds, fullerenes, and the like. They can be used singularly, or in a combination of two or more.

As used herein, the term “aromatic cyclic” compounds can refer to a substituted or unsubstituted C₆-C₅₀ cyclic aromatic system including one or more aromatic rings in which the rings may be attached together in a pendent manner or may be fused. Exemplary aromatic cyclic compounds useful in the present invention can include without limitation tetracene, anthracene, perylene, rubrene, and the like, and combinations thereof.

As used herein the term “heterocyclic” compounds can refer to a C₃-C₃₀ aromatic or alicyclic system consisting of one to three heteroatoms selected from N, O, P and S atoms and the remaining ring carbon atoms in which the rings may be attached together in a pendant manner or may be fused. Exemplary heterocyclic compounds useful in the present invention can include without limitation coumarin, and the like and combinations thereof.

The inorganic compound may include an ionic crystalline compound. As a non-limiting example, the inorganic compound may include a metal halide wherein the metal is selected from the group consisting of an alkali metal and an alkaline-earth metal. In one embodiment, the inorganic compound may be selected from the group consisting of sodium chloride, potassium chloride, lithium chloride, and mixtures thereof.

The first solvent is not limited as long as it can dissolve the organic or inorganic compound, it is not compatible with the second solvent when it is mixed with the second solvent, and it can form an emulsion by using the second solvent as a solvent phase so as to form a micro-droplet. Such solvents may include an organic solvent or an aqueous solvent.

When the compound for forming solid particulates is an organic compound, the first solvent can be an organic solvent having high solubility to the organic compound. Exemplary organic solvents useful as the first solvent for organic compounds can include without limitation aromatic hydrocarbon solvents such as toluene, xylene, monohalobenzene, dihalobenzene, trihalobenzene, and the like, aliphatic linear or cyclic hydrocarbon solvents such as hexane or cyclohexane, and the like, and organic halogen solvents such as carbon tetrachloride. The organic solvent may be used singularly, or two or more thereof may be mixed.

According to one embodiment, the organic solvent can have solubility to the organic compound ranging from about 1 mass % to a saturated concentration. When the solubility is less than about 1 mass %, the productivity of particles may be decreased. As used herein, the term “solubility” means a percentage of the amount of the solute included in the total amount of the solvent and solute, and the term “saturated concentration” means the solute concentration in a saturated solution.

When the compound for solid particulates is an inorganic compound, in one embodiment, the first solvent can be an aqueous solvent having high solubility to the inorganic compound. Exemplary aqueous solvents useful as the first solvent for inorganic compounds can include without limitation water such as ultra-pure water, highly purified water, purified water, deionized water, or tap water.

The aqueous solvent can have solubility to the inorganic compound ranging from about 1 mass % to a saturated concentration. When the solubility is less than about 1 mass %, the productivity of particles may be decreased.

According to one embodiment, the organic or inorganic compound-included solution can include an organic or inorganic compound in an amount ranging from about 0.1 mM to about 1000 mM, and in another embodiment, in an amount ranging from about 1.0 to about 100 mM. When the concentration of the organic or inorganic compound in the organic or inorganic compound-included solution is less than about 0.1 mM, insufficient nano-sized solid particulates may be formed. On the other hand, when the concentration of the organic or inorganic compound in the organic or inorganic compound-included solution is more than about 1000 mM, the resultant solid particulates may be aggregated with each other.

The organic or inorganic compound-included solution may further include any additive for desired purposes, such as to increase the solubility of the inorganic or organic compound in the first solvent.

The additive is not limited as long as it does not inhibit the physical properties of the particulates, and it remains in the dispersing medium. It may further include, for example, a cationic, anionic, or nonionic surfactant. The surfactant is not limited as long as it is commonly used as a cationic, anionic, or nonionic surfactant. The surfactant when present can be suitably selected based upon the kind of the organic or inorganic compound used.

The obtained organic or inorganic compound-included solution is dispersed in the second solvent to provide an emulsion (S2).

The second solvent is not limited as long as it is not compatible with the first solvent, and it can form an emulsion. When the compound for forming solid particulates is an organic compound, the second solvent may be included singularly or as a mixture of two or more aqueous solvents such as water. When the compound of solid particulates is an inorganic compound, the second solvent may be the above-mentioned organic solvent either singularly or as a mixture of two or more thereof.

According to one embodiment, the second solvent does not dissolve solid particulates, and in another embodiment, it has solubility to the solid particulates ranging from about 1×10⁻⁹ to about 1×10⁻⁸ mass % at 20° C.

The second solvent may further include an additive as necessary.

The additive may include an aggregation inhibitor that increases the dispersion property of micro-droplets to prevent aggregation. Exemplary aggregation inhibitors may include without limitation a cationic additive, an anionic additive, a zwitterionic additive, a nonionic additive, a polymer additive, and the like, and combinations thereof.

Exemplary cationic additives may include without limitation a quaternary ammonium salt, an aliphatic amine, an alkoxy polyamine, an aliphatic amine polyglycolether, a diamine, a polyamine derived from an aliphatic amine and an aliphatic alcohol, and the like, and combinations thereof.

Exemplary anionic additives may include without limitation a fatty acid salt, an alkylpolyphosphate ester, an alkylsulfate ester, an alkyl arylsulfonate, an arylsulfuric acid ester, acyl methyl taurinate, an alkylphosphate ester, an arylphosphate ester, an arylsulfonic acid formalin condensation, a polyoxyethylene alkylsulfate ester, and the like, and combinations thereof.

The zwitterionic additive is a compound having a cation structure of the cationic dispersing agent and an anionic structure of the anionic dispersing agent in one molecule.

Exemplary nonionic additives may include without limitation a polyoxyethylene fatty acid ester, a polyoxyethylene alkyl ether, a polyoxyethylene alkyl arylether, a polyoxyethylene alkylamine, a polyoxy alkyl arylamine, a polyoxyethylene fatty acid ester, a glycerine fatty acid ester, a sorbitan fatty acid ester, a polyoxyethylene sorbitan fatty acid ester, and the like, and combinations thereof.

Exemplary polymer additives may include without limitation alkylhydroxy cellulose, a cellulose derivative, polyvinylalcohol, polyvinylpyrrolidone, polyacrylate and derivatives thereof, an acetic acid vinyl copolymer, polyethyleneglycol, polypropyleneglycol, polyethyleneoxide, polycarbonate, polyvinylmethylether, polyacrylamide, polyimide, a polyarylamine salt, a polyethyleneoxy-polypropyleneoxide copolymer, a polyacrylic acid salt, a condensation arylene sulfonate, polyvinylsulfate, a styrene-acrylate copolymer, and the like, and combinations thereof.

According to one embodiment, such an additive can be added in an amount of about 0.001 to about 50 wt % based on the total weight of emulsion considering the aggregation of solid particulates.

The obtained organic or inorganic-included solution is added and mixed with the second solvent, and then the organic or inorganic-included solution forms micro-droplets (disperse phase) in the second solvent to provide an emulsion.

When the emulsion is prepared, the mixing ratio of the organic or inorganic compound-included solution and the second solvent is not limited. According to one embodiment, the mixing ratio of the organic or inorganic compound-included solution to the second solvent can be in a volume ratio ranging from about 0.01:1 to about 0.7:1, and in another embodiment, from about 0.05:1 to about 0.6:1. When the mixing ratio of the organic or inorganic compound-included solution to the second solvent is outside of this range, so the organic or inorganic compound-included solution is added in an excessively small amount relative to the second solvent, it may deteriorate the productivity of solid particulates; on the other hand, when the organic or inorganic compound-included solution is added in an excessively high amount relative to the second solvent, it can be difficult to form an emulsion.

The emulsion preparing process is not limited as long as the organic or inorganic compound-included solution is suitably mixed with the second solvent. According to one embodiment, they are mixed to provide a miniaturized dispersion phase. Exemplary emulsion preparing methods can include without limitation high-speed agitation, ultrasonic wave radiation, and a combination thereof. It is possible to provide a smaller emulsion by using high-speed mechanical agitation and a high power microwave radiator, so that it is possible to provide smaller organic particulates.

When the emulsion is prepared by high-speed agitation, the organic or inorganic compound-included solution and second solvent can be mechanically agitated at a rotation speed ranging from about 100 to about 30,000 rpm. In another embodiment, the organic or inorganic compound-included solution and second solvent can be mechanically agitated at a rotation speed ranging from about 500 to about 20,000 rpm. According to another embodiment, the agitating duration ranges from about 10 to about 60 minutes.

According to one embodiment, when the emulsion is prepared by ultrasonic wave radiation, it is irradiated with an ultrasonic wave having a frequency ranging from about 20 to about 45 kHz, and in another embodiment, it is radiated with an ultrasonic wave having a frequency ranging from about 25 to about 45 kHz. In another embodiment, the radiation duration ranges from about 1 to about 180 minutes, and in a further embodiment, it ranges from about 30 to about 60 minutes.

According to one embodiment, the emulsion can be prepared at a temperature up to the boiling point of the dispersing medium. For example, the emulsion can prepared at a temperature ranging from about 0 to about 100° C. when water is used as the dispersing medium.

The average diameter of droplets formed in the emulsion prepared by the method is not limited. According to one embodiment, the average diameter can range from about 100 nm to about 5 μm, and in another embodiment, the average diameter can range from about 100 nm to about 1 μm. Forming the droplets in the emulsion with a diameter within these size ranges can be useful because the emulsion size is not changed. As used herein, unless it is stated otherwise, the term “drop diameter” means the value measured by using a dynamic optical scattering method, and the term “average droplet diameter” means a numerical average diameter.

The organic or inorganic compound is precipitated into a solid particulate by concentrating the emulsion including micro-droplets in the second solvent in a dispersing medium to provide a dispersion including solid particulates (S3).

The process of concentrating the emulsion may be performed under reduced pressure.

The concentration speed is not limited as long as the organic or inorganic compound particulates are precipitated. In an embodiment, the concentration can be performed while reducing pressure at a rate ranging from about 0.01 to about 10.0 hPa/min, and in further another embodiment, the concentration can be performed while reducing pressure at a rate of about 0.1 to about 5.0 hPa/min. If the concentration is performed by reducing the pressure at the above-mentioned rate, it is possible to provide uniform solid particulates, and the concentration is facilitated by driving a vacuum device to provide the reduced pressure. The method to reduce the pressure is not limited, and it can be readily determined depending upon the pressure range for reducing pressure and the reactor scale.

Furthermore, the associated organic solvent and the temperature conditions during the concentration may be further considered, in addition to the concentration speed.

With regard to temperature conditions, the solute can be dissolved in the solvent at a relatively higher temperature when the first solvent has low solubility. Therefore, the solute can be mixed in the dispersing medium at the same temperature to provide an emulsion, and it is then concentrated. When the temperature is higher, a smaller emulsion is obtained resulting in the particulates having a smaller diameter.

The device for reducing the pressure is not limited, and it may include a rotary evaporator using a pressure shifter mounted with a vacuum pump.

As a result of concentration due to the pressure reduction, the organic or inorganic compound included in the emulsion in droplets is precipitated in the form of solid particulates, so the emulsion becomes a dispersion.

A physical or chemical external stimulus may be further applied in order to facilitate the precipitation of solid particulates in addition to the concentration process. The physical external stimulus may include a temperature change. When the emulsion is prepared at room temperature, removal of the solvent by concentrating the emulsion at a high temperature is more beneficial to precipitate solid particulates. The high temperature means a temperature of the boiling point or less of the dispersing medium at the predetermined reduced pressure condition.

The chemical external stimulus may be an additive such as an antifoaming agent. The antifoaming agent may include any one as long as it is conventionally used as an antifoaming agent, and it may include any common antifoaming agents commercially available in the market.

The amount of solid particulates prepared by the method is not limited. In one embodiment, when the solid particulates are prepared in the dispersion at a high concentration, the concentration of particulates can range from about 1.0 to about 1000 μg/ml immediately after the particulates are precipitated after completing the concentration of the emulsion. According to another embodiment, the concentration of particulates can range from about 5.0 to about 500 μg/ml. As used herein, the term “concentration of solid particulates” means the value of the mass of solid particulates included in the dispersion in which the organic or inorganic compound is precipitated as particulates divided by the total volume of dispersion.

It is possible to provide a particulate dispersion having a high concentration even though the method of the invention does not require a complicated condensation process of a dispersing system due to the method of preparing the solid particulates, and it does not include unnecessary solvents.

Thereafter, the dispersing medium is removed from the obtained dispersion in accordance with a general method such as filtration and drying to provide solid particulates dispersed in the dispersion.

According to one embodiment, the solid particulates prepared by the method can have a nanometer-sized average diameter; in another embodiment the solid particulates prepared by the method can have an average diameter ranging from about 10 to about 1000 nm; and in another embodiment, the solid particulates prepared by the method can have an average diameter ranging from about 30 to about 500 nm. As used herein, unless stated otherwise, the term “diameter of solid particulate” means a value measured in accordance with the dynamic optical scattering method, and the term “average diameter” means a numerical average diameter.

The solid particulates prepared by the method are prepared as almost perfect crystals having a smaller lattice bond than that of micro-grain particles prepared by conventional precipitation methods. This is because the conventional re-precipitation accomplishes non-parallel crystal growth in which crystal growing is completed within several milliseconds. In contrast, the method according to the present invention can provide high quality micro-crystals since it accomplishes parallel crystal growth in which the crystals grow within several minutes to several hours.

The good solvent remaining in the particulate dispersion obtained from the conventional re-precipitation cannot be removed in principle. Evaporation of the dispersing medium and the remaining good solvent by drying can be considered. However, in this case, the particulates may aggregate. Furthermore, because the remaining good solvent cannot be removed by a bubbling method using nitrogen gas in the conventional method, it is impossible to use the obtained particulate dispersion in the medical field. In contrast, in the present invention, the organic solvent as opposed to the particulates and the dispersing medium does not remain as shown by results of observing the particulate dispersion prepared from the inventive method using a proton nuclear magnetic resonance device.

As the re-precipitation uses a polar solvent having relative low solubility toward the organic compound, the organic particulates have a narrow size-control range. In contrast, the present invention can control the size over a wide range since the present inventive method uses a non-polar organic solvent having high solubility toward the organic compound.

The solid particulates prepared by the method according to one embodiment of the present invention can be used in a color filter for a display, particularly in a case of nano-organic solid particulates.

The following examples are suggested for facilitating understanding of the present invention, but the present invention is not limited to the following examples.

### EXAMPLE 1

20 mL of ultra-pure water (resistivity: 18.2 MΩ·cm) is introduced into a 100 mL flask, and 2 mL of a solution (1.06 mM) in which perylene is added to a mixed solvent of toluene/cyclohexane (=3:7 volume ratio) is added to the flask. It is then agitated with a mechanical agitator (Tokyo Rikakikai Co., LTD., NE-1000 type) at 800 rpm and simultaneously treated with ultrasonic waves (45 kHz) for approximately 5 minutes to provide an emulsion. Droplets included in the emulsion have a diameter of 400 nm.

Subsequently, the obtained emulsion is positioned in a rotary evaporator mounted with a pressure shifter adjusted to be 130 hPa in the system and then concentrated at a reducing pressure rate of 1.5 hPa/min. When the reduced pressure reaches about 80 hPa, it provides 110 mL of an aqueous dispersion of perylene solid particulates.

The diameter of the obtained perylene solid particulates is measured with a dynamic optical scattering photometer (Otsuka Electronics Co., Ltd., FPAR-1000). As shown by the results, the obtained perylene solid particulate has an average diameter of 180 nm.

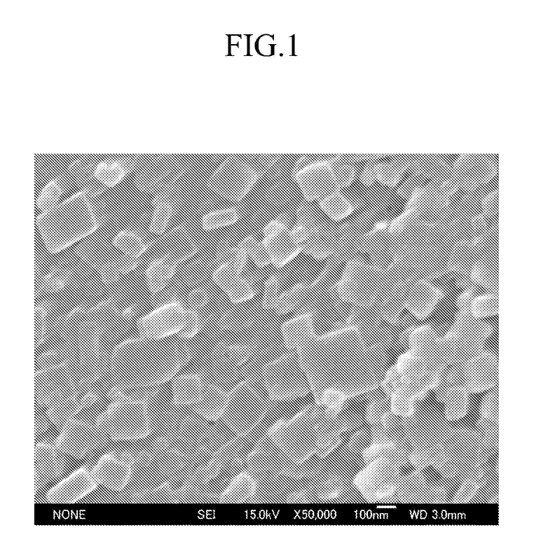

Furthermore, the obtained perylene solid particulate is observed (magnification: 50,000) with a scanning electron microscope. The results are shown in FIG. 1.

It is confirmed that the size of the perylene solid particulates is controlled to a nano-size.

### EXAMPLE 2

An aqueous dispersion in which perylene solid particulates are dispersed is prepared in accordance with the same procedure as in Example 11 except that a solution in which perylene having a concentration of 2.12 mM is dissolved in a toluene/cyclohexane (=3:7 volume ratio) solvent.

### EXAMPLE 3

An aqueous dispersion in which perylene solid particulates are dispersed is prepared in accordance with the same procedure as in Example 1, except that a solution in which perylene having a concentration of 3.33 mM is dissolved in a toluene/cyclohexane (=3:7 volume ratio) solvent.

### EXAMPLE 4

An aqueous dispersion that perylene solid particulates are dispersed is prepared in accordance with the same procedure as in Example 1, except that a solution in which perylene having a concentration of 4.69 mM is dissolved in a toluene/cyclohexane (=3:7 volume ratio) solvent.

### COMPARATIVE EXAMPLE 1

5 ml of ultra-pure water is added to 5 ml of a tetrahydrofuran-saturated solution of perylene to provide a water-included perylene solution. Nitrogen gas is flowed to the perylene solution at a flowing rate of 0.2 L/min for 1 hour to remove tetrahydrofuran, so as to provide 5 mL of a perylene aqueous dispersion.

Generally, the dispersion stability becomes higher as the zeta potential value decreases. Thereby, the particulate aqueous dispersions of Example 1 and Comparative Example 1 are measured for zeta potential to determine dispersion stability.

The measurement results show that the particulate aqueous dispersion of Example 1 has a zeta potential of −40 mV, while the particulate aqueous dispersion according to Comparative Example 1 has a high zeta potential of 30 mV. The organic solvent remaining in the aqueous dispersion increases the zeta potential of the aqueous dispersion of Comparative Example 1. It is understood that the particulates obtained from the method according to the present invention have excellent dispersion stability.

In addition, the particulate aqueous dispersions of Example 1 and Comparative Example 1 are allowed to stand at room temperature for 70 hours with shielding, and the particulate change in the particulate aqueous dispersion is observed.

The measurement results show that the perylene nano-crystals in the particulate aqueous dispersion of Example 1 did not change as time passed, while the perylene particulate in the particulate aqueous dispersion of the comparative example is seen to have an increasing particle diameter.

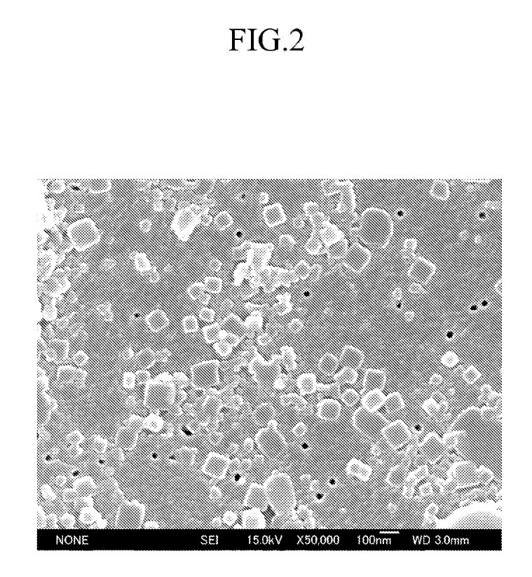

The solid particulates obtained from Examples 2 to 4 are observed with a scanning electron microscope (magnification: 50,000). The results are shown in FIGS. 2 to 4.

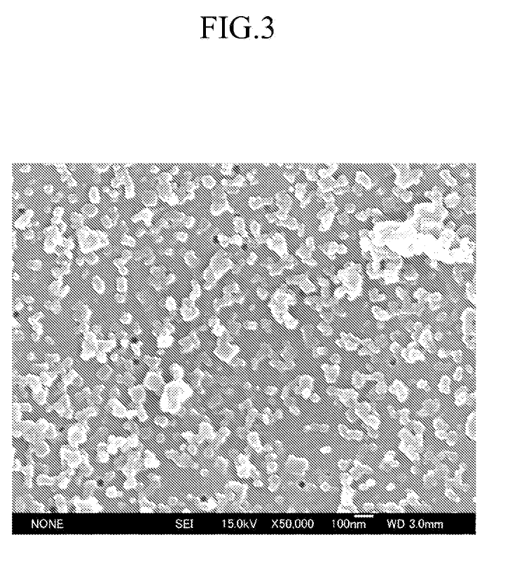

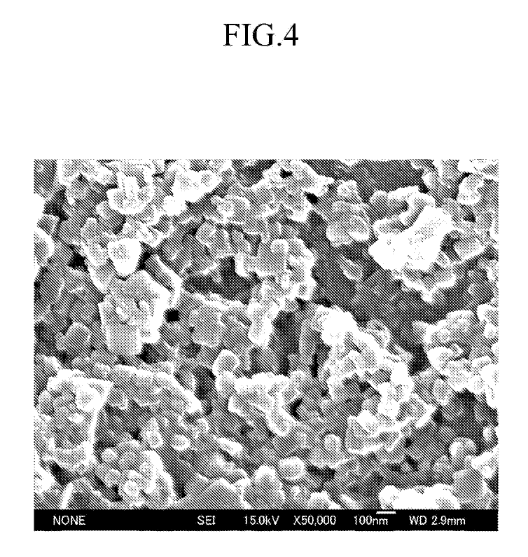

FIG. 2 shows a scanning electron microscope (SEM) photograph of perylene particulates obtained from Example 2 of the present invention, FIG. 3 shows a scanning electron microscope (SEM) photograph of perylene particulates obtained from Example 3 of the present invention, and FIG. 4 shows a scanning electron microscope (SEM) photograph of perylene particulates obtained from Example 4 of the present invention.

As shown in FIGS. 2 to 4, the results of the scanning electron microscope photographs show that the average diameter of the perylene solid particulates obtained from Example 2 is 100 nm, and that the average diameters of the perylene solid particulates obtained from Examples 3 and 4 are 60 nm and 90 nm, respectively. From the results, it is recognized that solid particulates having a nano-size according to the present invention were prepared in a dispersion.

Furthermore, the solid particulate aqueous dispersions obtained from Examples 1 to 4 are allowed to stand at room temperature for 6 months with shielding, and it is observed whether the precipitate is generated.

The results show that the precipitate is observed in none of the solid particulate dispersions of Examples 1 to 4.

The first solvent is evaluated as to whether it remains in the aqueous dispersion of solid particulate obtained from Examples 1 to 4 by using the ultra-violet visible absorption spectrum (JASCO Corporation, V-550).

From the measurement results, the first solvent of cyclohexane and toluene is not confirmed in any of the solid particulate dispersions of Examples 1 to 4. Accordingly, it is demonstrated that the desirable solid particulate having a simply controlled size is dispersed in the dispersing medium, and that it is possible to provide a solid particulate having high dispersion stability.

The amounts of solid particulates included in the solid particulate dispersion of Examples 1 to 4 are measured. The results are shown in the following Table 1.

TABLE 1

| | Example 1 | Example 2 | Example 3 | Example 4 |
|---|---|---|---|---|
| Concentration | 27 | 53 | 84 | 118 |
| of solid | | | | |
| particulates | | | | |
| (μg/Ml) | | | | |

As shown in Table 1, it is possible to provide solid particulates having a high concentration with the preparation method of the present invention.

Many modifications and other embodiments of the invention will come to mind to one skilled in the art to which this invention pertains having the benefit of the teachings presented in the foregoing descriptions. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation, the scope of the invention being defined in the claims.

What is claimed is:

1. A method of preparing solid particulates comprising:
dissolving an organic or inorganic compound in a first solvent to provide an organic or inorganic compound-included solution;
dispersing the organic or inorganic compound-included solution in a second solvent to provide an emulsion; and
concentrating the emulsion in a dispersing medium to precipitate the organic or inorganic compound as a solid particulate so as to provide a dispersion comprising solid particulates,
wherein the first solvent is an organic solvent or an aqueous solvent and the second solvent is either an organic solvent or an aqueous solvent that is not compatible with the first solvent.

2. The method of claim 1, wherein the concentrating step is performed by reducing pressure.

3. The method of claim 1, wherein the organic compound is selected from the group consisting of aromatic cyclic compounds, heterocyclic compounds, fullerenes, and mixtures thereof.

4. The method of claim 1, wherein the inorganic compound is an ionic crystalline compound.

5. The method of claim 1, wherein the organic solvent has solubility to the organic compound ranging from about 1 mass % to a saturated concentration.

6. The method of claim 1, wherein the aqueous solvent has solubility to the inorganic compound ranging from about 1 mass % to a saturated concentration.

7. The method of claim 1, wherein the second solvent has solubility to the solid particulates of about 1.0×10⁻¹ g/L or less at 20° C.

8. The method of claim 1, wherein the organic or inorganic compound-included solution is mixed with the second solvent at a volume ratio ranging from about 0.01:1 to about 0.7:1.

9. The method of claim 1, wherein the step of preparing an emulsion is performed by one selected from the group consisting of high-speed agitation of the organic or inorganic compound-included solution, ultrasonic wave radiation, and a combination thereof.

10. The method of claim 1, wherein the concentrating step is performed by reducing the pressure of the emulsion to a range of about 0.01 to about 10.0 hPa/min.

11. The method of claim 1, wherein the concentrating step is performed by reducing the pressure of the emulsion to a range of about 0.1 to about 5.0 hPa/min.

12. The method of claim 1, wherein the dispersion comprises solid particulates in a concentration ranging from about 1.0 to about 1000 μg/ml.

13. Solid particulates prepared using the method of claim 1.

14. The solid particulates of claim 13, wherein the solid particulates have an average diameter ranging from about 10 to about 1000 nm.
